(19) United States
(12) Patent Application Publication
(10) Pub. No.: US 2022/0202553 A1
(43) Pub. Date: Jun. 30, 2022

(54) EMBOLIC PROTECTION DEVICE

(71) Applicants: Ram Nozyk, Jerusalem (IL); TEL HASHOMER MEDICAL RESEARCH INFRASTRUCTURE AND SERVICES LTD., Ramat-Gan (IL)

(72) Inventors: Ram Nozyk, Jerusalem (IL); Moshe Halak, Zerufa, Carmel Coast (IL)

(21) Appl. No.: 17/604,037

(22) PCT Filed: Apr. 16, 2020
(86) PCT No.: PCT/IB2020/053604

§ 371 Date: Oct. 15, 2021

(30) Foreign Application Priority Data
Apr. 18, 2019 (GB) 1905551.6

(51) Int. Cl. A61F 2/01; A61M 29/00

(52) U.S. Cl. CPC A61F 2/014; A61F 2230/0067; A61M 29/00; A61F 2/011

(57) ABSTRACT

Apparatus and methods are described for capturing emboli during a medical procedure that is performed at a first location within a subjects body. An intravascular device (24) is configured to perform the medical procedure at the first location. An inner sheath (32) facilitates insertion of the intravascular device (24) into the subjects body, by the intravascular device being inserted via a lumen (31) of the inner sheath (32). A filter (34) extending from a distal-most portion (42) of the inner sheath (32) has a non-radially-constrained configuration in which the filter (34) is configured to prevent at least some emboli that are generated by the medical procedure from passing through the filter (34). Other applications are also described.

### CROSS-REFERENCES TO RELATED APPLICATIONS

The present application claims priority from U.K. Patent Application 1905551.6, filed Apr. 18, 2019, entitled “Embolic Protection Device,” which is incorporated herein by reference.

### FIELD OF EMBODIMENTS OF THE INVENTION

The present invention relates to medical apparatus and methods, and specifically to apparatus and methods for capturing emboli.

### BACKGROUND

Intravascular procedures can release particulate debris in blood vessels. Particulate debris in the vascular system contribute to complications including vascular occlusion and end-organ ischemia. Catheters used in percutaneous procedures are typically characterized as defining a relatively large ratio between the length and the diameter of the catheter. Typically, due to the catheter length being much greater than the diameter of the catheter, there is a degree of instability at the distal end of the catheter, which is disposed within the patient's vasculature. In arterial procedures, the catheter is typically inserted in a retrograde direction, against blood which is flowing at a high flow rate. The instability of the distal end of the catheter, in combination with the high flow rate against the direction of the catheter's advancement, often causes the catheter to strike the walls of the artery.

Critical care patients, upon whom such procedures are typically performed, are already at increased risk due to pre-existing comorbidities such as age and other diseases. Therefore, such patients are particularly vulnerable to emboli being released into the arterial blood stream, during the advancement of the catheter. It is common for emboli that are released during such procedures to flow to the stomach, the intestines, liver, kidneys and lower limbs.

Various ways to filter such emboli from the vascular system before they arrive at distal organs have been proposed. Embolic protection devices have increased the safety of percutaneous interventions. Filters deployed in the arterial system capture particulate debris in the blood.

In many transcatheteral procedures, an introducer sheath is used to facilitate placing the catheter through a percutaneous incision into a vein or artery. Percutaneous introducer sheaths are used to facilitate both the initial percutaneous introduction, and/or for the exchange of intravascular devices. The introducer sheath is typically used in combination with a dilator. The dilator is typically relatively stiff and has a pointed tip. During the percutaneous insertion of the sheath, the dilator is typically disposed inside the sheath, with the pointed tip protruding from the distal end of the introducer sheath. The pointed tip of the dilator dilates the percutaneous incision, and the stiffness of the dilator supports the sheath during the insertion of the sheath through the incision. Typically, the dilator is removed after positioning the sheath inside the blood vessel. The sheath is then left in the blood vessel, such as to hold open the incision during the procedure and to protect the vessel from trauma.

### SUMMARY OF EMBODIMENTS

In accordance with some applications of the present invention, an embolic protection device is placed inside a subject's vasculature downstream of a location at which a transcatheteral procedure is being performed. Typically, the embolic protection device is used during transcatheteral procedures in which an intravascular device, such as a catheter, is introduced via a percutaneous incision, and then via the subject's arterial system. Typically, the catheter is used to perform a procedure within the subject's aorta, coronary arteries, carotid arteries, and/or within the subject's heart. For example, such procedures may include deployment of a prosthetic valve, deployment of a stent, deployment of a stent graft, an atherectomy procedure, an angioplasty procedure, and/or any other procedure that is used by a person skilled in the art.

As described in the Background section hereinabove, in such procedures, the catheter is inserted in a retrograde direction, against blood that is flowing at a high flow rate. The instability of the distal end of the catheter, in combination with the high flow rate against the direction of the catheter's advancement often causes the catheter to strike the walls of the artery, thereby releasing emboli. In the absence of embolic protection device, the emboli that are released typically flow to the subject's lower body. Therefore, in accordance with some applications of the present invention, an embolic protection device is first deployed within the subject's vasculature downstream of the location at which the medical procedure is to be performed. Subsequently, the intravascular device is inserted into the subject's vasculature, and the procedure is performed using the intravascular device. Upon completion of the procedure, the intravascular device is retracted from the subject's body.

It is noted that the embolic protection device described herein is typically configured for use with any intravascular device, such as, an intravascular tool (e.g., a wire that is used to apply an intravascular therapy), and/or a percutaneous catheter. However, the embolic protection device is typically used with an intravascular device that is used in the performance of a medical procedure, as opposed to a guidewire, for example, which is typically used for guiding other tools to a location at which the other tools perform a procedure. The embolic protection device is typically configured to provide embolic protection during the advancement and retraction of the intravascular device, as well as during the procedure itself. Further typically, the embolic protection device additionally functions (a) to provide a lumen via which the intravascular device (e.g., the catheter) is inserted through the percutaneous incision, and (b) to hold open the percutaneous incision during the procedure.

Although some applications are described herein as being applied to a subject's arterial vasculature, the scope of the present invention includes applying the apparatus and methods described herein to procedures that are performed on the venous side of subject's vasculature, mutatis mutandis.

The term “distal” and related terms, when used with reference to a device or a portion thereof, should be interpreted to mean an end of the device or the portion thereof that, when inserted into a subject's body, is typically further from the location through which the device is inserted into the subject's body. The term “proximal” and related terms, when used with reference to a device or a portion thereof, should be interpreted to mean an end of the device or the portion thereof that, when inserted into a subject's body, is typically closer to the location through which the device is inserted into the subject's body. The terms “downstream” and “upstream” and related terms should be interpreted as being defined with respect to the direction of antegrade blood flow through a subject's vasculature. Thus, by way of example, the descending aorta is downstream of the ascending aorta, and the descending aorta of upstream of the iliac arteries.

There is therefore provided, in accordance with some applications of the present invention, a method for capturing emboli during a medical procedure that is performed at a first location within a body of a subject, the method including:

placing a filter within vasculature of the subject at a second location that is downstream of the first location,
the filter extending from a distal-most portion of an inner sheath that is shaped to define a lumen therethrough, and
the placing of the filter at the second location being performed by advancing the filter to the second location while the filter is maintained in a radially-constrained configuration within an outer sheath;

when the filter is disposed at the second location, causing the filter to assume a non-radially-constrained configuration, in which the filter is configured to prevent at least some emboli that are generated by the medical procedure from passing through the filter;

subsequently, advancing an intravascular device to the first location via the lumen defined by the inner sheath, and performing the medical procedure at the first location using the intravascular device; and

subsequently, sliding at least one of the inner sheath and the outer sheath with respect to the other, such that the filter becomes disposed within the outer sheath, such as to cause the filter to assume the radially-constrained configuration with at least some of the emboli that are generated by the procedure becoming trapped within the filter.

In some applications, performing the medical procedure at the first location using the intravascular device includes performing the medical procedure using the intravascular device at a first location that is at least 5 cm from the second location at which the filter is deployed.

In some applications, the first location at which the medical procedure is performed is selected from the group consisting of: an aortic arch, a carotid artery, a subclavian artery, an ascending aorta, a thoracic aorta, and a location within a heart of the subject, and causing the filter to assume the non-radially-constrained configuration when the filter is disposed at the second location includes causing the filter to assume the non-radially-constrained configuration in an abdominal aorta of the subject.

In some applications, causing the filter to assume the non-radially-constrained configuration when the filter is disposed at the second location includes causing the filter to assume the non-radially-constrained configuration in an abdominal aorta of the subject.

In some applications, the first location at which the medical procedure is performed is selected from the group consisting of: an aortic arch, a carotid artery, a subclavian artery, an ascending aorta, a thoracic aorta, and a location within a heart of the subject.

In some applications, the first location at which the medical procedure is performed is selected from the group consisting of: an aortic arch, a carotid artery, a subclavian artery, an ascending aorta, a thoracic aorta, and a location within a heart of the subject, and causing the filter to assume the non-radially-constrained configuration when the filter is disposed at the second location includes causing the filter to assume the non-radially-constrained configuration in an iliac artery of the subject.

In some applications, causing the filter to assume the non-radially-constrained configuration when the filter is disposed at the second location includes causing the filter to assume the non-radially-constrained configuration in an iliac artery of the subject.

In some applications, performing the medical procedure at the first location using the intravascular device includes performing a medical procedure selected from the group consisting of: deployment of a prosthetic valve, deployment of a stent, deployment of a stent graft, an atherectomy procedure, and an angioplasty procedure.

In some applications, the intravascular device includes a catheter and performing the medical procedure at the first location using the intravascular device includes performing the medical procedure at the first location using the catheter.

In some applications, the outer sheath includes an introducer sheath, and placing the filter within the subject's vasculature at the second location that is downstream of the first location includes advancing the inner sheath and the filter through the introducer sheath.

In some applications, causing the filter to assume the non-radially-constrained configuration includes causing the filter to assume a non-radially-constrained configuration in which the filter widens radially from a proximal end of the filter to a location that is distal thereto.

In some applications, causing the filter to assume the non-radially-constrained configuration includes causing the filter to assume a non-radially-constrained configuration in which at least a portion of the filter is shaped as a funnel.

In some applications, causing the filter to assume the non-radially-constrained configuration includes causing the filter to assume a non-radially-constrained configuration in which the filter defines a wide portion that contacts an inner wall of a blood vessel in which the filter is deployed.

In some applications, causing the filter to assume the non-radially-constrained configuration includes causing the filter to assume a non-radially-constrained configuration in which there is at least some overlap between the filter and the distal region of the inner sheath, such that a volume between the filter and the distal region of the inner sheath defines a reservoir for the emboli.

In some applications, an outer diameter of the inner sheath at the distal region of the inner sheath is smaller than the outer diameter of the inner sheath at locations of the inner sheath that are proximal to the distal region of the inner sheath.

In some applications, the distal region of the inner sheath has a flexibility that is such that when the filter is disposed within the outer sheath, the distal region of the inner sheath at least partially collapses radially.

In some applications, sliding at least one of the inner sheath and the outer sheath with respect to the other, such that the filter becomes disposed within the outer sheath, such as to cause the filter to assume a radially-constrained configuration in which at least some of the emboli that are generated by the procedure become trapped within the filter includes causing a distal region of the inner sheath to at least partially collapse radially, such that the emboli are accommodated within a space between the filter and the distal region of the inner sheath.

In some applications,

placing the filter within the subject's vasculature at the second location includes placing the filter within the subject's vasculature at the second location while a dilator is disposed within the lumen defined by the inner sheath,

the dilator defining a proximal region having a first diameter, and a pointed tip, and

the dilator being disposed within the lumen defined by the inner sheath with the pointed tip of the dilator at least partially protruding from a distal end of the inner sheath.

In some applications,

the dilator further defines a narrow-diameter region disposed between the proximal region and the pointed tip, the narrow-diameter region having a diameter that is smaller than the first diameter, and

placing the filter within the subject's vasculature at the second location includes placing the filter within the subject's vasculature at the second location with the filter disposed within the outer sheath, and with the narrow diameter region of the dilator overlapping longitudinally with the filter.

In some applications:

causing the filter to assume the non-radially-constrained configuration includes causing the filter to assume a non-radially-constrained configuration in which there is at least some overlap between the filter and the distal region of the inner sheath, such that a volume between the filter and the distal region of the inner sheath defines a reservoir for the emboli, and

sliding at least one of the inner sheath and the outer sheath with respect to the other, such that the filter becomes disposed within the outer sheath, such as to cause the filter to assume a radially-constrained configuration in which at least some of the emboli that are generated by the procedure become trapped within the filter includes causing a distal region of the inner sheath to at least partially collapse radially against the narrow-diameter region of the dilator, such that the emboli are accommodated within a space between the filter and the distal region of the inner sheath.

In some applications, advancing the intravascular device to the first location via the lumen defined by the inner sheath includes advancing the intravascular device through a unidirectional valve disposed at a proximal end of the inner sheath.

In some applications, advancing the intravascular device through the unidirectional valve disposed at a proximal end of the inner sheath includes advancing the intravascular device through a hemostasis valve disposed at a proximal end of the inner sheath.

There is further provided, in accordance with some applications of the present invention, apparatus for capturing emboli during a medical procedure that is performed at a first location within a body of a subject, the apparatus including:

an inner sheath that is shaped to define a lumen therethrough;

an intravascular device that is configured to be insertable into the subject's body, by being advanced through the lumen defined by the inner sheath, the intravascular device being configured to perform the medical procedure at the first location within the subject's body;

a filter extending from a distal-most portion of the inner sheath, the filter being configured to be deployed at a second location that is downstream of the first location, and the filter having a non-radially-constrained configuration in which the filter is configured to prevent at least some emboli that are generated by the medical procedure from passing through the filter; and

an outer sheath disposed outside at least a portion of the inner sheath, the outer sheath being configured to cause the filter to assume a radially-constrained configuration in which at least some of the emboli that are generated by the procedure become trapped within the filter, by at least one of the inner and outer sheaths being slid with respect to the other, such that the filter becomes disposed within the outer sheath.

In some applications, the intravascular device is configured to perform the medical procedure at the first location, the first location being at least 5 cm from the second location at which the filter is deployed.

In some applications, the first location is a location selected from the group consisting of: an aortic arch, a carotid artery, a subclavian artery, an ascending aorta, a thoracic aorta, and a location within a heart of the subject, and the second location is an abdominal aorta of the subject.

In some applications, the first location is a location selected from the group consisting of: an aortic arch, a carotid artery, a subclavian artery, an ascending aorta, a thoracic aorta, and a location within a heart of the subject, and the second location is an iliac artery of the subject.

In some applications, the intravascular device is configured to perform a medical procedure selected from the group consisting of: deployment of a prosthetic valve, deployment of a stent, deployment of a stent graft, an atherectomy procedure, and an angioplasty procedure.

In some applications, the outer sheath includes an introducer sheath.

In some applications, the intravascular device includes a catheter that is configured to be insertable into the subject's body, by being advanced through the lumen defined by the inner sheath.

In some applications, the filter is configured to prevent emboli that are generated during the advancement of the catheter, during a transcatheteral procedure performed using the catheter, and during retraction of the catheter.

In some applications, in its non-radially-constrained configuration, the filter widens radially from a proximal end of the filter to a location that is distal thereto.

In some applications, in its non-radially-constrained configuration, at least a portion of the filter is shaped as a funnel.

In some applications, in its non-radially-constrained configuration, the filter defines a wide portion that is configured to contact an inner wall of a blood vessel in which the filter is deployed.

In some applications, the filter extends from the distal-most portion of the inner sheath such that there is at least some overlap between the filter and the distal region of the inner sheath, such that a volume between the filter and the distal region of the inner sheath defines a reservoir for the emboli.

In some applications, an outer diameter of the inner sheath at the distal region of the inner sheath is smaller than the outer diameter of the inner sheath at locations of the inner sheath that are proximal to the distal region of the inner sheath.

In some applications, the distal region of the inner sheath has a flexibility that is such that when the filter is disposed within the outer sheath, the distal region of the inner sheath at least partially collapses radially.

In some applications, the distal region of the inner sheath has a flexibility that is such that when the filter is disposed within the outer sheath with emboli disposed inside the reservoir between the filter and distal region, the distal region of the inner sheath at least partially collapses radially, such that the emboli are accommodated within a space between the filter and the distal region.

In some applications, the apparatus further includes a dilator that defines a proximal region having a first diameter, and a pointed tip, the dilator is configured to be disposed inside the lumen defined by the inner sheath with the pointed tip of the dilator at least partially protruding from a distal end of the inner sheath, at least during insertion of the embolic protection device into the subject's body.

In some applications, the dilator further defines a narrow-diameter region disposed between the proximal region and the pointed tip, the narrow-diameter region having a diameter that is smaller than the first diameter, and the narrow diameter region being configured to overlap longitudinally with the filter when the filter is disposed within the outer sheath.

In some applications:

the filter extends from the distal-most portion of the inner sheath such that there is at least some overlap between the filter and the distal region of the inner sheath, such that a volume between the filter and the distal region of the inner sheath defines a reservoir for the emboli, and

the distal region of the inner sheath has a flexibility that is such that when the filter is disposed within the outer sheath with emboli disposed inside the reservoir between the filter and distal region, the distal region of the inner sheath at least partially collapses radially against the narrow-diameter region of the dilator, such that the emboli are within a space between the filter and the distal region.

In some applications, the apparatus further includes a unidirectional valve disposed at a proximal end of the inner sheath.

In some applications, the unidirectional valve includes a hemostasis valve.

There is further provided, in accordance with some applications of the present invention, apparatus for capturing emboli during a medical procedure that is performed at a first location within a body of a subject, the apparatus being for use with an introducer sheath, the apparatus including:

an inner sheath that is shaped to define a lumen therethrough;

an intravascular device that is configured to be insertable into the subject's body, by being advanced through the lumen defined by the inner sheath, the intravascular device being configured to perform the medical procedure at the first location within the subject's body;

a filter extending from a distal-most portion of the inner sheath, the filter being configured to be deployed at a second location that is downstream of the first location, and the filter having a non-radially-constrained configuration in which the filter is configured to prevent at least some emboli that are generated by the medical procedure from passing through the filter,

the filter being configured such that the filter assumes a radially-constrained configuration in which at least some of the emboli that are generated by the procedure become trapped within the filter, by the introducer sheath and the inner sheath being slid with respect to the other, such that the filter becomes disposed within the introducer sheath.

There is further provided, in accordance with some applications of the present invention, apparatus for capturing emboli during a medical procedure that is performed at a first location within a body of a subject, the apparatus including:

a sheath that is shaped to define a lumen therethrough;

an intravascular device that is configured to be insertable into the subject's body, by being advanced through the lumen defined by the inner sheath, the intravascular device being configured to perform the medical procedure at the first location within the subject's body;

a filter disposed at a distal end of the sheath,

the filter being configured to be deployed at a second location that is downstream of the first location, and the filter being configured to prevent at least some emboli that are generated by the medical procedure from traversing the second location.

There is further provided, in accordance with some applications of the present invention, a device for capturing emboli during a medical procedure that is performed at a first location within a body of a subject, the device including:

an inner sheath that is shaped to define a lumen therethrough;

a filter extending from a distal-most portion of the inner sheath, the filter being configured to be deployed at a second location that is downstream of the first location, and the filter having a non-radially-constrained configuration in which the filter is configured to prevent at least some emboli from passing through the filter; and

an outer sheath disposed outside at least a portion of the inner sheath, the outer sheath being configured to cause the filter to assume a radially-constrained configuration in which emboli become trapped within the filter, by at least one of the inner and outer sheaths being slid with respect to the other, such that the filter becomes disposed within the outer sheath,

an intravascular device, that is inserted into the subject's body during the medical procedure, is configured to be insertable by being advanced through the lumen defined by the inner sheath, the intravascular device being configured to perform the medical procedure at the first location within the subject's body.

There is further provided, in accordance with some applications of the present invention, a method for capturing emboli during a medical procedure, the medical procedure being performed at a first location within a body of a subject, the method including:

(i) introducing a device for capturing emboli during the medical procedure, the device including an inner sheath, a filter and an outer sheath, wherein:
the inner sheath is shaped to define a lumen therethrough;
the filter extends from a distal-most portion of the inner sheath, the filter being configured to be deployed at a second location downstream of the first location, and the filter having a non-radially-constrained configuration; and
the outer sheath is disposed outside at least a portion of the inner sheath;

(ii) placing the filter at the second location, while the filter is maintained in a radially-constrained configuration within the outer sheath;

(iii) causing the filter to assume a non-radially-constrained configuration, in which the filter is configured to prevent at least some emboli from passing through the filter;

(iv) inserting an intravascular device to perform the medical procedure at the first location, wherein the intravascular device is inserted by being advanced through the lumen defined by the inner sheath, wherein emboli are captured during the procedure by the non-radially-constrained filter; and

(v) causing the filter to assume a radially-constrained configuration by sliding at least one of the inner sheath or the outer sheath with respect to the other, such that the filter becomes disposed within the outer sheath, with at least some of the emboli that are generated by the procedure becoming trapped within the filter.

The present invention will be more fully understood from the following detailed description of applications thereof, taken together with the drawings, in which:

### BRIEF DESCRIPTION OF THE DRAWINGS

FIGS. 1A and 1B are schematic illustrations of an embolic protection device placed inside a subject's vasculature downstream of a location at which a transcatheteral procedure is being performed, in accordance with some applications of the present invention;

FIGS. 2A, 2B, 2C, and 2D are schematic illustrations of portions of an embolic protection device, in accordance with some applications of the present invention;

FIGS. 3A, 3B, 3C, 3D, and 3E are schematic illustrations of the embolic protection device at respective stages of a procedure that is performed using the embolic protection device, in accordance with some applications of the present invention.

### DETAILED DESCRIPTION OF EMBODIMENTS

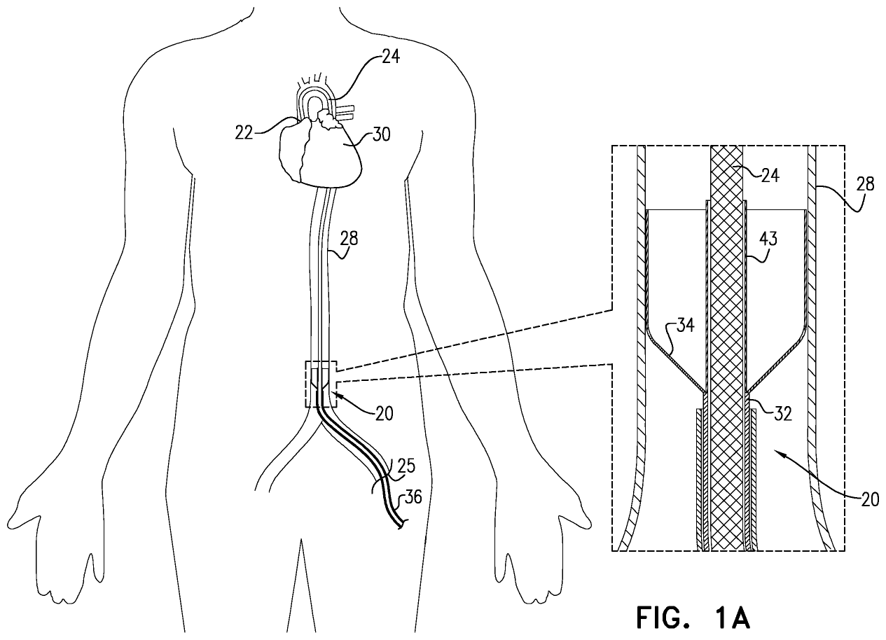

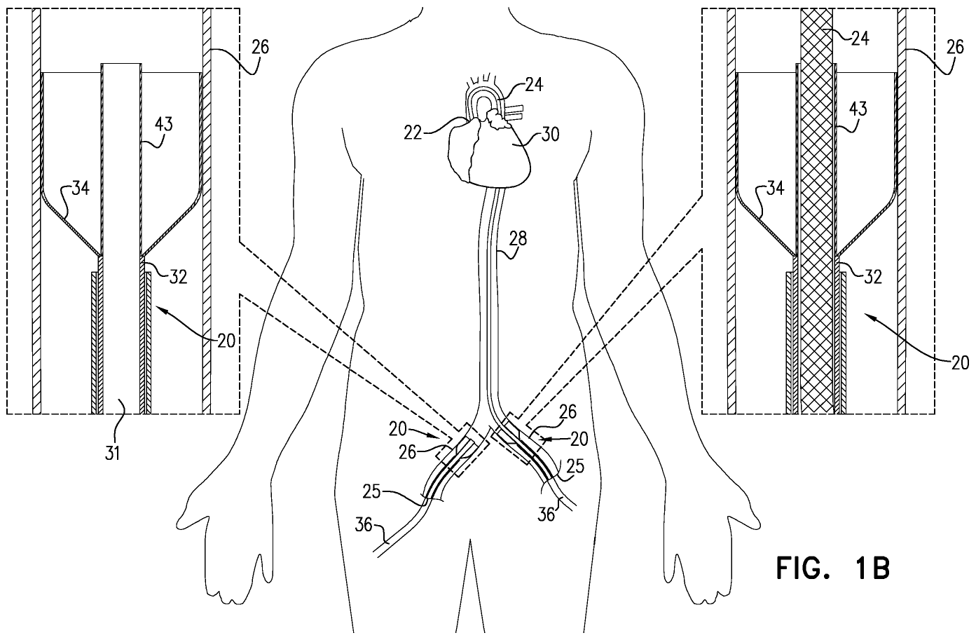

Reference is now made to FIGS. 1A and 1B, which are schematic illustrations of an embolic protection device 20 placed inside a subject's vasculature downstream of a location 22 at which a transcatheteral procedure is being performed, in accordance with some applications of the present invention. Typically, the embolic protection device is used during transcatheteral procedures, in which an intravascular device, such as a catheter, is introduced via the subject's arterial system. For example, as shown in FIGS. 1A and 1B, catheter 24 has been inserted via the subject's femoral artery 25, and has been advanced through the subject's aorta 28 toward the subject's heart 30. Typically, the catheter is used to perform a procedure within the subject's aorta (e.g., thoracic aorta, ascending aorta, and/or aortic arch), coronary arteries, carotid arteries, and/or within the subject's heart. For example, such procedures may include deployment of a prosthetic valve, deployment of a stent, deployment of a stent graft, an atherectomy procedure, an angioplasty procedure, and/or another procedure that is known in the art.

As described in the Background section hereinabove, in such procedures, the catheter is inserted in a retrograde direction, against blood that is flowing at a high flow rate. The instability of the distal end of the catheter, in combination with the high flow rate against the direction of the catheter's advancement often causes the catheter to strike the walls of the artery, thereby releasing emboli. In the absence of embolic protection device 20, the emboli that are released typically flow to the subject's lower body. Therefore, in accordance with some applications of the present invention, embolic protection device is deployed within the subject's vasculature downstream of location 22, at which the transcatheteral procedure is to be performed. Subsequently, catheter 24 is inserted into the subject's vasculature, and the transcatheteral procedure is performed using the catheter. Upon completion of the procedure, the catheter is retracted from the subject's body.

It is noted that the embolic protection device described herein is typically configured for use with any percutaneous catheter. It is further noted that, while some aspects of the present invention are described with respect to catheter 24 being used as the intravascular device, the scope of the present invention includes using embolic protection device 20 with any intravascular working device (e.g., a wire that is used to apply an intravascular therapy), mutatis mutandis. However, the embolic protection device is typically used with an intravascular device that is used in the performance of a medical procedure, as opposed to a guidewire, for example, which is typically used for guiding other tools to a location at which the other tools perform a procedure.

As described herein, the embolic protection device is typically configured to provide embolic protection during the advancement and retraction of the intravascular device, as well as during the procedure itself. Further typically, the embolic protection device additionally functions (a) to provide a lumen via which the intravascular device is inserted through the percutaneous incision, and (b) to hold open the percutaneous incision during the procedure. For example, FIGS. 1A and 1B show catheter 24 being inserted via a lumen 31 (shown in FIG. 1B), defined by an inner sheath 32 of the embolic protection device, and outer sheath 36 of the embolic protection device holding open the percutaneous incision. Embolic protection device 20 is typically is configured to block at least some emboli that are released either during the advancement of the catheter, during the transcatheteral procedure, and/or during retraction of the catheter, and typically substantially all of the released emboli, or all of the released emboli, from passing a filter 34 of the embolic protection device (the filter being described in further detail hereinbelow). Typically, subsequent to the catheter being retracted, the filter of the embolic protection device is radially constrained such as to trap at least some of the released emboli, and typically substantially all of the released emboli, or all of the released emboli, within the filter. The embolic protection device, with the trapped emboli disposed therein, is then retracted from the subject's body.

As shown in FIG. 1A, for some applications, a single embolic protection device 20 is deployed within the subject's aorta 28 (e.g., the subject's abdominal aorta). In this manner, the embolic protection device is configured to protect both sides of the subject's lower body from emboli that are released during the procedure. For some applications, embolic protection device 20 is deployed within the aorta at a location that is superior to the mesenteric arteries, the renal arteries, the gastric arteries, and/or the hepatic arteries, such as to provide embolic protection to the colon, the kidneys, the stomach, and/or the liver, in addition to arteries of the lower body.

Alternatively, as shown in FIG. 1B, first and second embolic protection devices are deployed, respectively, within right-sided and left-sided arteries, e.g., right and left iliac arteries 26, in order to provide lower-body protection to the respective sides of the subject's body. For some applications, only one of the embolic protection devices is used to provide percutaneous access to catheter 24, while the second embolic protection device is used solely to provide the embolic protection function. For example, as shown in FIG. 1B, catheter 24 is inserted via the left-sided embolic protection device, whereas there are no devices inserted via lumen 31 defined by inner sheath 32 of the right-sided embolic protection device. Alternatively, both embolic protection devices are used to facilitate percutaneous insertion of devices.

As noted above, typically, the intravascular device, e.g., catheter 24, is configured to perform a medical procedure. It is typically the case that the intravascular device performs the medical procedure at a location that is remote from the location at which filter 34 is deployed, for example, a location that is at least 5 cm (e.g., at least 10 cm or 15 cm) upstream of the location at which the filter is deployed. The intravascular device is typically advanced to the location at which the medical procedure is to be performed by being advanced (e.g., slid) through inner lumen 31 defined by the inner sheath 32 of the embolic protection device.

It is noted that although some applications are described herein as being applied to a subject's arterial vasculature, the scope of the present invention includes applying the apparatus and methods described herein to procedures that are performed on the venous side of subject's vasculature, mutatis mutandis.

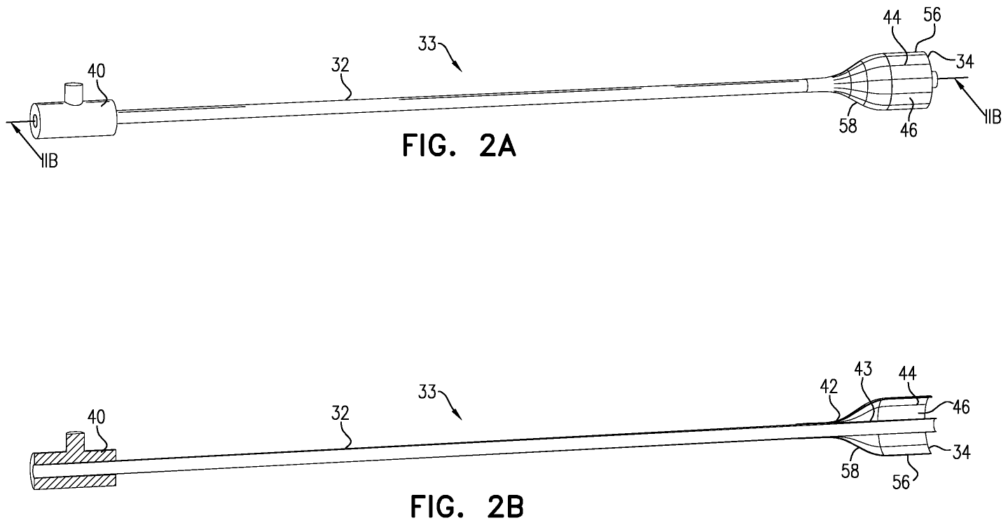

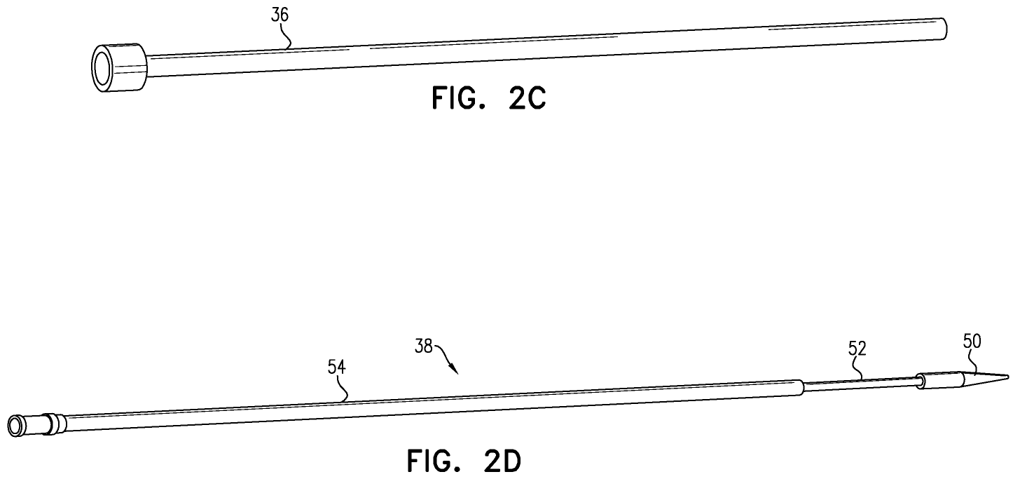

Reference is now made to FIGS. 2A, 2B, 2C, and 2D, which are schematic illustrations of portions of embolic protection device 20, in accordance with some applications of the present invention. FIGS. 2A and 2B are schematic illustrations of, respectively, a side view, and a cross-section view, of a filter unit 33 of the embolic protection device, in accordance with some applications of the present invention. FIG. 2C is a schematic illustration of an outer sheath 36 of the embolic protection device, in accordance with some applications of the present invention. FIG. 2D is a schematic illustration of a dilator 38 that is used as part of the embolic protection device, in accordance with some applications of the present invention.

Referring to FIGS. 2A and 2B, filter unit 33 typically includes inner sheath 32, which is shaped to define lumen 31 therethrough. The inner sheath is thereby configured to facilitate insertion of catheter 24 (or a different intravascular device) into the subject's body, by the intravascular catheter being inserted via the lumen. For some applications, the filter unit additionally includes a unidirectional valve 40 (e.g., a hemostasis valve) at the proximal end of the inner sheath. The unidirectional valve is configured prevent lumen 31 from becoming a channel for blood flowing out of the body. Alternatively or additionally, the distal end of the inner sheath is tapered (not shown), such that the tapered distal end (a) prevents blood from flowing between the inner sheath and catheter 24, and (b) prevents blood from flowing through the distal end of the inner sheath when catheter 24 is withdrawn from inside the inner sheath.

Typically, filter 34 extends from a distal location 42 of the inner sheath. It is noted that, for some applications, the filter does not extend from the tip of the inner sheath. Rather, the filter extends from a location that is toward the distal tip of the inner sheath, but slightly proximal therefrom, such that there is at least some overlap between the filter and a distal region 43 of the inner sheath. The filter typically has a non-radially-constrained configuration, in which the filter is configured to prevent emboli that are generated by the medical procedure from passing through the filter. Typically, while the filter is in its non-radially-constrained configuration, inside the blood vessel, the volume between filter 34 and distal region 43 of inner sheath 32 acts as a reservoir for the emboli. For some applications (not shown), the outer diameter of inner sheath 32 at distal region 43 is smaller than at locations of the inner sheath that are proximal to region 43, such that the reservoir between filter 34 and distal region 43 of inner sheath 32 is larger than if the distal region were to have the same outer diameter as locations of the inner sheath that are proximal to region 43.

Typically, in its non-radially-constrained configuration, filter 34 is shaped such that the filter widens radially from the proximal end of the filter (i.e., the end of the filter that is coupled to the inner sheath) to a location that is distal thereto, such that the filter defines a relatively wide portion 56 and a relatively narrow portion 58. For example, the filter may be shaped such that its distal end is wider than its proximal end, and/or such that at least a portion of the filter is shaped as a funnel, as shown. For some applications, the widening of the filter is such that the outer wall of the filter contacts the inner surface of the blood vessel in which the filter is deployed. For some applications, the filter includes a shape-memory material 44 (e.g., a shape-memory alloy, such as nitinol), which is shape-set such that the filter automatically assumes its non-radially-constrained shape, in the absence of any forces acting upon the filter. Typically, the shape-memory material is covered with a covering material 46 (e.g., a fabric and/or a polymer, such as polyurethane, polytetrafluoroethylene (PTFE), expanded polytetrafluoroethylene (ePTFE), or woven, knitted and/or braided polyester). Further typically, the covering material is permeable with respect to blood, such that the subject's blood traverses the filter, but is impermeable with respect to at least some emboli, such that the emboli are prevented from traversing the filter, and passing into the subject's bloodstream downstream of the filter. Typically, the covering material is impermeable at least with respect to emboli that are of a sufficient size to potentially cause clinical damage. For some applications, the covering material has a pore size of more than 20 microns (e.g., more than 70 microns), and/or less than 300 microns (e.g., less than 200 microns), e.g., 20-300 microns, or 70-200 microns.

Referring now to FIG. 2C, outer sheath 36 is typically sized such that the inner diameter of the outer sheath is approximately equal to (or slightly greater than) the outer diameter of inner sheath 32. For some applications, the outer sheath has a generally similar configuration to a standard introducer sheath, as is known in the art. For some applications, outer sheath 36 is a standard introducer sheath, as is known in the art, and filter unit 33, and/or dilator 38 are configured to be used with the standard introducer sheath. Typically, the inner sheath is disposed inside the outer sheath, and catheter 24 is introduced via lumen 31 defined by inner sheath. The diameter of lumen 31 is typically 0.2 mm-0.8 mm (e.g., approximately 1-3 French) less than the lumen defined by the outer sheath. Thus, introducing the catheter via lumen 31 defined by inner sheath 32 typically reduces the effective working diameter by a relatively small amount, such as, 0.2 mm-0.8 mm (e.g., approximately 1-3 French), relative to if the catheter were to be introduced directly via the lumen defined by the outer sheath.

Typically, outer sheath 36 is slidable with respect to inner sheath 32, and vice vera. Further typically, the outer sheath is configured to cause filter 34 to assume a radially-constrained configuration, by at least one of the outer sheath and inner sheath being slid with respect to the other, such that the filter becomes disposed within the outer sheath. For some applications, the outer sheath maintains the filter in its radially-constrained configuration, during insertion of embolic protection device 20 into the subject's body. When the distal-most portion of the inner sheath is disposed in the vicinity of the location at which the filter is to be deployed, at least one of the outer sheath and the inner sheath is slid with respect to the other (typically, by advancing the inner sheath with respect to the outer sheath), such that the filter is no longer disposed inside the outer sheath. The filter thereby assumes its non-radially-constrained configuration.

Typically, subsequent to the transcatheteral procedure being performed and catheter 24 having been withdrawn from the subject's body, the outer sheath causes at least some of the emboli that are released by the procedure, and typically substantially all of the released emboli, or all of the released emboli, to become trapped within the filter. At least one of the inner and outer sheaths is typically slid with respect to the other, such that the filter becomes disposed within the outer sheath, and assumes its radially-constrained configuration with the emboli disposed inside the filter. As described hereinabove, for some applications, distal region 43 of inner sheath 32 overlaps with filter 34, such there is a reservoir between the filter and distal region 43, within which the emboli become trapped. For some applications, distal region 43 of inner sheath 32 is sufficiently flexible that when the filter is radially constrained by outer sheath 36 with emboli disposed inside the reservoir between the filter and distal region 43, distal region 43 at least partially collapses radially, such that the emboli can be accommodated within the space between the filter and distal region 43. Typically, the flexibility of distal region 43 of inner sheath 32 is also such that, when the filter is radially constrained by outer sheath 36 during insertion of embolic protection device into the subject's body, distal region 43 at least partially collapses radially. Further typically, the filter is at least partially accommodated around the outside of the collapsed distal region, thereby reducing the profile (i.e. diameter) of embolic protection device 20 relative to if distal region 43 did not collapse radially.

Referring now to FIG. 2D, in some applications, dilator 38 is placed inside lumen 31 defined by inner sheath 32, during insertion of embolic protection device 20 into the subject's body. The dilator typically has a pointed tip 50 and is typically stiffer than the inner and outer sheaths. The pointed tip of the dilator is used to dilate the incision via which embolic protection device 20 is inserted, and the stiffness of the dilator supports the inner and outer sheaths during the insertion of the sheaths through the incision.

Alternatively or additionally, the dilator is placed inside lumen 31 defined by inner sheath 32, after the transcatheteral procedure has been performed, and prior to the outer sheath and inner sheath being slid with respect to slid with respect to the other, such that the filter becomes disposed within the outer sheath. For some applications, the dilator includes a narrow-diameter region 52, which has a diameter that is less than the diameter of a proximal region 54 of the dilator, which is proximal to the narrow-diameter region. For example, a ratio of the diameter of the dilator proximal to the narrow-diameter region, to the diameter of the narrow-diameter region may be between 1:5 and 1:20.

Typically, the narrow-diameter region is configured to be positioned with respect to inner sheath 32, such that when the filter is radially constrained by outer sheath 36, there is longitudinal overlap between the filter and the narrow-diameter region of the dilator, and such that the filter is disposed around the narrow-diameter region. For some applications, distal region 43 of inner sheath 32 is sufficiently flexible that when the filter is radially constrained by outer sheath 36, distal region 43 collapses against the narrow-diameter region of the dilator. Thus, during insertion of the embolic protection device, the profile (i.e., diameter) of the radially-constrained device is reduced relative to if the dilator did not include the narrow-diameter region. Moreover, as described hereinabove, for some applications, distal region 43 of inner sheath 32 overlaps with filter 34, such there is a reservoir between the filter and distal region 43, within which the emboli become trapped. For some applications, distal region 43 of inner sheath 32 is sufficiently flexible that when the filter is radially constrained by outer sheath 36 with emboli disposed inside the reservoir between the filter and distal region 43, distal region 43 collapses against the narrow-diameter region of the dilator, such that the emboli can be accommodated within the space between the filter and distal region 43.

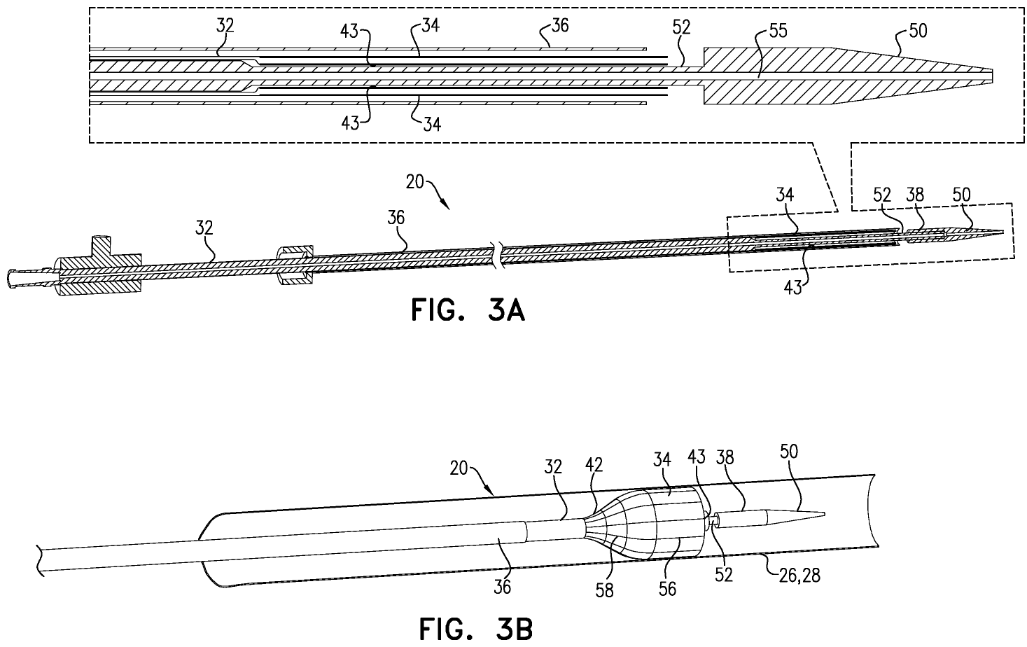

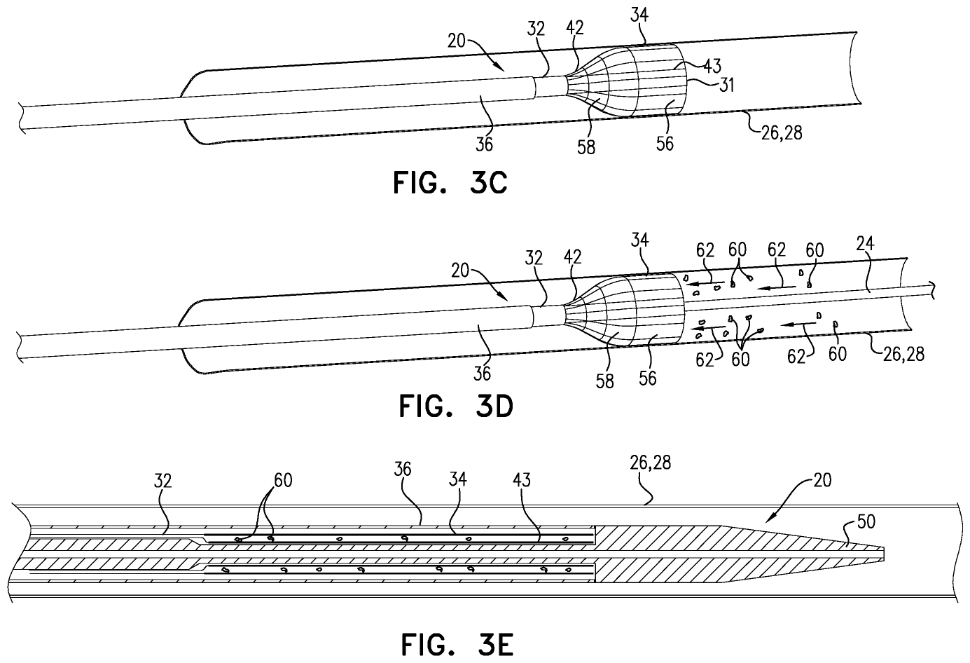

Reference is now made to FIGS. 3A, 3B, 3C, 3D, and 3E, which are schematic illustrations of embolic protection device 20 at respective stages of a procedure that is performed using the embolic protection device, in accordance with some applications of the present invention.

Referring to FIG. 3A, prior to the insertion of the embolic protection device into the subject's body, outer sheath 36 is positioned with respect to inner sheath 32, such that filter 34 is disposed inside the outer sheath and is maintained in its radially-constrained configuration by the outer sheath. For some applications, dilator 38 is disposed inside lumen 31 defined by inner sheath 32, with pointed tip 50 of the dilator at least partially protruding distally from the inner and outer sheaths. As described hereinabove, the pointed tip of the dilator is typically used to dilate the incision via which embolic protection device 20 is inserted, and the stiffness of the dilator supports the inner and outer sheaths during the insertion of the sheaths through the incision. As shown in FIG. 3A, typically the dilator defines a guidewire lumen 55 therethrough, to facilitate insertion of the dilator over a guidewire.

Referring to FIG. 3B, in order to deploy filter 34, at least one of the outer sheath and inner sheath is slid with respect to the other (typically, by advancing the inner sheath with respect to the outer sheath), such that the filter is no longer disposed within the outer sheath, and such that the filter assumes its non-radially-constrained configuration. FIG. 3B shows embolic protection device, after the filter has assumed its non-radially-constrained configuration within an artery (such as, iliac artery 26, or aorta 28), such that wide portion 56 of the filter is in contact with the inner wall of the artery. As shown in FIG. 3B, dilator 38 has not yet been retracted from lumen 31 defined by inner sheath 32.

Referring now to FIG. 3C, subsequent to deployment of filter 34, the dilator is typically retracted, such that the working tools for the percutaneous procedure can be inserted via lumen 31 defined by inner sheath 32. FIG. 3C shows the embolic protection device with the dilator having been retracted, but prior to the working tools having been inserted through lumen 31.

Referring to FIG. 3D, subsequent to retraction of dilator 38, catheter 24 is inserted via lumen 31 defined by inner sheath 32. As schematically illustrated in FIG. 3D, emboli 60 that are released during the advancement of catheter 24, during retraction of catheter 24, and/or during the procedure performed using catheter 24, flow toward filter 34 in the direction of arrows 62. The emboli are prevented from traversing the filter, such that emboli are held within the reservoir between filter 34 and distal region 43 of inner sheath 32.

Referring now to FIG. 3E, subsequent to the percutaneous procedure having been performed, catheter 24 is retracted from lumen 31 defined by inner sheath 32. At least one of the outer sheath and the inner sheath is then slid with respect to the other, such that the filter is radially constrained by the outer sheath, such that at least some of the emboli becomes trapped within the filter. For some application, prior to the outer sheath radially constraining the filter, dilator 38 is inserted via lumen 31. Alternatively, the outer sheath radially constrains the filter, in the absence of dilator 38.

It is generally noted that although some applications of the present invention have described embolic protection device 20 as including dilator 38, the scope of the present invention includes using the apparatus and methods described herein in the absence of the dilator. For example, the outer sheath may be used to radially constrain the filter and to thereby trap the emboli between the filter and distal region 43 of the inner sheath, even in the absence of dilator 38.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method for capturing emboli during a medical procedure that is performed at a first location within a body of a subject, the method comprising:
placing a filter within vasculature of the subject at a second location that is downstream of the first location,
the filter extending from a distal-most portion of an inner sheath that is shaped to define a lumen therethrough, and
the placing of the filter at the second location being performed by advancing the filter to the second location while the filter is maintained in a radially-constrained configuration within an outer sheath;
when the filter is disposed at the second location, causing the filter to assume a non-radially-constrained configuration, in which the filter is configured to prevent at least some emboli that are generated by the medical procedure from passing through the filter;
subsequently, advancing an intravascular device to the first location via the lumen defined by the inner sheath, and performing the medical procedure at the first location using the intravascular device; and
subsequently, sliding at least one of the inner sheath and the outer sheath with respect to the other, such that the filter becomes disposed within the outer sheath, such as to cause the filter to assume the radially-constrained configuration with at least some of the emboli that are generated by the procedure becoming trapped within the filter.

2. The method according to claim 1, wherein performing the medical procedure at the first location using the intravascular device comprises performing the medical procedure using the intravascular device at a first location that is at least 5 cm from the second location at which the filter is deployed.

3. The method according to claim 1, wherein the first location at which the medical procedure is performed is selected from the group consisting of: an aortic arch, a carotid artery, a subclavian artery, an ascending aorta, a thoracic aorta, and a location within a heart of the subject, and wherein causing the filter to assume the non-radially-constrained configuration when the filter is disposed at the second location comprises causing the filter to assume the non-radially-constrained configuration in an abdominal aorta of the subject.

4-5. (canceled)

6. The method according to claim 1, wherein the first location at which the medical procedure is performed is selected from the group consisting of: an aortic arch, a carotid artery, a subclavian artery, an ascending aorta, a thoracic aorta, and a location within a heart of the subject, and wherein causing the filter to assume the non-radially-constrained configuration when the filter is disposed at the second location comprises causing the filter to assume the non-radially-constrained configuration in an iliac artery of the subject.

7. (canceled)

8. The method according to claim 1, wherein performing the medical procedure at the first location using the intravascular device comprises performing a medical procedure selected from the group consisting of: deployment of a prosthetic valve, deployment of a stent, deployment of a stent graft, an atherectomy procedure, and an angioplasty procedure.

9. (canceled)

10. The method according to claim 1, wherein the outer sheath includes an introducer sheath, and wherein placing the filter within the subject's vasculature at the second location that is downstream of the first location comprises advancing the inner sheath and the filter through the introducer sheath.

11-12. (canceled)

13. The method according to claim 1, wherein causing the filter to assume the non-radially-constrained configuration comprises causing the filter to assume a non-radially-constrained configuration in which the filter defines a wide portion that contacts an inner wall of a blood vessel in which the filter is deployed.

14. The method according to claim 1, wherein causing the filter to assume the non-radially-constrained configuration comprises causing the filter to assume a non-radially-constrained configuration in which there is at least some overlap between the filter and the distal region of the inner sheath, such that a volume between the filter and the distal region of the inner sheath defines a reservoir for the emboli.

15-17. (canceled)

18. The method according to claim 1,
wherein placing the filter within the subject's vasculature at the second location comprises placing the filter within the subject's vasculature at the second location while a dilator is disposed within the lumen defined by the inner sheath,
the dilator defining a proximal region having a first diameter, and a pointed tip, and
the dilator being disposed within the lumen defined by the inner sheath with the pointed tip of the dilator at least partially protruding from a distal end of the inner sheath.

19-22. (canceled)

23. Apparatus for capturing emboli during a medical procedure that is performed at a first location within a body of a subject, the apparatus comprising:
an inner sheath that is shaped to define a lumen therethrough;
an intravascular device that is configured to be insertable into the subject's body, by being advanced through the lumen defined by the inner sheath, the intravascular device being configured to perform the medical procedure at the first location within the subject's body;
a filter extending from a distal-most portion of the inner sheath, the filter being configured to be deployed at a second location that is downstream of the first location, and the filter having a non-radially-constrained configuration in which the filter is configured to prevent at least some emboli that are generated by the medical procedure from passing through the filter; and
an outer sheath disposed outside at least a portion of the inner sheath, the outer sheath being configured to cause the filter to assume a radially-constrained configuration in which at least some of the emboli that are generated by the procedure become trapped within the filter, by at least one of the inner and outer sheaths being slid with respect to the other, such that the filter becomes disposed within the outer sheath.

24-28. (canceled)

29. The apparatus according to claim 23, wherein the intravascular device comprises a catheter that is configured to be insertable into the subject's body, by being advanced through the lumen defined by the inner sheath.

30. The apparatus according to claim 29, wherein the filter is configured to prevent emboli that are generated during the advancement of the catheter, during a transcatheteral procedure performed using the catheter, and during retraction of the catheter.

31. The apparatus according to claim 23, wherein, in its non-radially-constrained configuration, the filter widens radially from a proximal end of the filter to a location that is distal thereto.

32-33. (canceled)

34. The apparatus according to claim 23, wherein the filter extends from the distal-most portion of the inner sheath such that there is at least some overlap between the filter and the distal region of the inner sheath, such that a volume between the filter and the distal region of the inner sheath defines a reservoir for the emboli.

35. The apparatus according to claim 34, wherein an outer diameter of the inner sheath at the distal region of the inner sheath is smaller than the outer diameter of the inner sheath at locations of the inner sheath that are proximal to the distal region of the inner sheath.

36. The apparatus according to claim 34, wherein the distal region of the inner sheath has a flexibility that is such that when the filter is disposed within the outer sheath, the distal region of the inner sheath at least partially collapses radially.

37. The apparatus according to claim 34, wherein the distal region of the inner sheath has a flexibility that is such that when the filter is disposed within the outer sheath with emboli disposed inside the reservoir between the filter and distal region, the distal region of the inner sheath at least partially collapses radially, such that the emboli are accommodated within a space between the filter and the distal region.

38. The apparatus according to claim 23, further comprising a dilator that defines a proximal region having a first diameter, and a pointed tip, wherein the dilator is configured to be disposed inside the lumen defined by the inner sheath with the pointed tip of the dilator at least partially protruding from a distal end of the inner sheath, at least during insertion of the embolic protection device into the subject's body.

39. The apparatus according to claim 38, wherein the dilator further defines a narrow-diameter region disposed between the proximal region and the pointed tip, the narrow-diameter region having a diameter that is smaller than the first diameter, and the narrow diameter region being configured to overlap longitudinally with the filter when the filter is disposed within the outer sheath.

40. The apparatus according to claim 39, wherein:
the filter extends from the distal-most portion of the inner sheath such that there is at least some overlap between the filter and the distal region of the inner sheath, such that a volume between the filter and the distal region of the inner sheath defines a reservoir for the emboli, and
the distal region of the inner sheath has a flexibility that is such that when the filter is disposed within the outer sheath with emboli disposed inside the reservoir between the filter and distal region, the distal region of the inner sheath at least partially collapses radially against the narrow-diameter region of the dilator, such that the emboli are within a space between the filter and the distal region.

41-46. (canceled)
